(19) United States
(12) Patent Application Publication
(10) Pub. No.: US 2020/0371182 A1
(43) Pub. Date: Nov. 26, 2020

(54) CALCULATION OF CONTRAST AGENT CONCENTRATION

(71) Applicant: Siemens Healthcare GmbH, Erlangen (DE)

(72) Inventors: Robert Grimm, Nuernberg (DE); Marcel Dominik Nickel, Herzogenaurach (DE)

(73) Assignee: Siemens Healthcare GmbH, Erlangen (DE)

(21) Appl. No.: 16/880,691

(22) Filed: May 21, 2020

(30) Foreign Application Priority Data
May 24, 2019 (DE) 102019207639.4

(51) Int. Cl. G01R 33/56; A61B 5/055; G01R 33/50

(52) U.S. Cl. CPC G01R 33/5601; G01R 33/50; A61B 5/055

(57) ABSTRACT

A method for determining a contrast agent concentration can be performed after contrast agent administration based on acquired MR images taken before and after contrast agent administration. The method can include the automated steps of determining a model equation describing the contrast agent concentration as a function of a plurality of model parameters, determining values for the plurality of model parameters taking the acquired MR images into account, determining the contrast agent concentration based on the model equation and the values determined, checking whether the contrast agent concentration determined is within an expected value range. If not, determining a corrected contrast agent concentration on the basis of the model equation and a corrected value for at least one of the model parameters. The corrected value for this at least one model parameter is determined such that the contrast agent concentration having the corrected value is within the expected value range.

### CROSS REFERENCE TO RELATED APPLICATIONS

This patent application claims priority to German Patent Application No. 102019207639.4, filed May 24, 2019, which is incorporated herein by reference in its entirety.

### BACKGROUND

### Field

The present disclosure relates to a method for determining a contrast agent concentration in an object under examination after contrast agent administration and to a method for determining at least one pharmacokinetic parameter of a pharmacokinetic model. Also provided is the associated device for determining the contrast agent concentration or pharmacokinetic parameter, a computer program product and an electronically readable data medium.

### Related Art

For T1-weighted dynamic contrast-enhanced magnetic resonance imaging (DCE-MRI), a gadolinium-based contrast agent is injected into the person under examination. The magnetic resonance (MR) signal measurements are carried out and repeated while the contrast agent reaches a target region in the body. The contrast agent reduces the T1 relaxation time and is also absorbed by different types of tissue at different rates. For example, the signal rise in hypervascularized tissue such as tumors is greater than in the surrounding healthy tissue.

Pharmacokinetic models attempt to explain quantitatively the observed signal changes over time, e.g. using a compartmental model. The observed signal difference or relative signal gain relative to the signal intensity before contrast agent administration is used to estimate the change in relaxivity caused by the contrast agent. This is in turn proportional to the contrast agent concentration. This relationship also depends on the imaging sequence used to acquire the MR images before, during and after contrast agent administration. For T1-weighted images, this is usually a gradient echo sequence, wherein the contrast agent concentration is estimated as follows:

$$C ( t ) = \frac{1}{r 1} \cdot \frac{Δ S ( t ) \cdot R 1 ( 0 ) \cdot ( 1 - cos θ + R 1 ( 0 ) \cdot cos θ \cdot TR )}{1 - cos θ - Δ S ( t ) \cdot R 1 ( 0 ) \cdot cos θ \cdot TR} \qquad ( 1 )$$

where R1 is the specific relaxivity of the contrast agent, ΔS(t) is the relative signal change at time t, R1(0) is the relaxation rate before contrast agent administration with R1(0)=1/T10, TR is the reaction time and θ is the flip angle of the imaging sequence used. For this calculation of the contrast agent concentration on the basis of the signal change, it is necessary to know model parameters such as e.g. the T1 relaxation rate, the repetition time TR, the flip angle and the relaxivity of the contrast agent.

The assumptions concerning precise knowledge of these parameters are often inapplicable because, for example, the effective flip angle can vary due to B1 field inhomogeneities, and additional time-consuming MR measurements must be carried out for accurate T1 measurements. A typical assumption is that the flip angle is constant over the entire image, and often a constant T1 time is also specified. This results in errors in determining the contrast agent concentration. Even if the T1 time of the tissue being examined is determined on the basis of the acquired MR images, this determination is still 30 to 40% subject to error. From the contrast agent concentration it is then possible to determine pharmacokinetic parameters which make it possible to assess whether the tissue examined is benign or malignant.

A particularly undesirable consequence of the above-mentioned disadvantages is that the denominator of the above equation (1) can become zero or become negative e.g. under the following condition:

$$Δ S ( t ) \geq \frac{1 - cos ( θ )}{R 1 cos θ \cdot TR} \qquad ( 2 )$$

This means that for very high relative signal increases and if, for example, R1 has been over-estimated, i.e. T1 has been estimated too small, there is a possibility that the contrast agent concentration will become negative, which makes no sense physiologically and is particularly problematic, as the entire pharmacokinetic model is inoperative in such high perfusion regions.

As the pharmacokinetic models are used to create parameter cards concerning the pharmacokinetic model parameters, these cards provide no reliable information as to the strength of the signal change caused by the contrast agent.

Negative contrast agent concentrations have hitherto been deemed to be incorrect and then simply set to zero, or the entire concentration curve has been set to zero. In all these cases, the estimated pharmacokinetic model parameters do not reflect the high perfusion level in the associated regions.

### BRIEF DESCRIPTION OF THE DRAWINGS/FIGURES

The accompanying drawings, which are incorporated herein and form a part of the specification, illustrate the embodiments of the present disclosure and, together with the description, further serve to explain the principles of the embodiments and to enable a person skilled in the pertinent art to make and use the embodiments.

FIG. 1 schematically illustrates an MR system and a device for determining contrast agent concentrations or pharmacokinetic parameters according to an exemplary embodiment.

FIG. 2 schematically illustrates how negative contrast agent concentrations would result as a function of different Ti times according to an exemplary embodiment.

FIG. 3 schematically illustrates how negative contrast agent constellations can be prevented by reducing the maximum signal intensity change according to an exemplary embodiment.

FIG. 4 schematically illustrates how negative concentrations can also be prevented by limit values for the T1 time according to an exemplary embodiment.

FIG. 5 is a flowchart of a method of determining the contrast agent concentration according to an exemplary embodiment.

FIG. 6 is a flowchart of a method for determining a pharmacokinetic parameter of a pharmacokinetic model according to an exemplary embodiment.

The exemplary embodiments of the present disclosure will be described with reference to the accompanying drawings. Elements, features and components that are identical, functionally identical and have the same effect are—insofar as is not stated otherwise—respectively provided with the same reference character.

### DETAILED DESCRIPTION

In the following description, numerous specific details are set forth in order to provide a thorough understanding of the embodiments of the present disclosure. However, it will be apparent to those skilled in the art that the embodiments, including structures, systems, and methods, may be practiced without these specific details. The description and representation herein are the common means used by those experienced or skilled in the art to most effectively convey the substance of their work to others skilled in the art. In other instances, well-known methods, procedures, components, and circuitry have not been described in detail to avoid unnecessarily obscuring embodiments of the disclosure. The connections shown in the figures between functional units or other elements can also be implemented as indirect connections, wherein a connection can be wireless or wired. Functional units can be implemented as hardware, software or a combination of hardware and software.

An object of the present disclosure is to eliminate the above-mentioned problems and reliably detect where hypervascularized regions occur after contrast agent administration in order to be able to gauge contrast model concentrations or other parameters of pharmacokinetic models.

According to a first aspect, a method for determining a contrast agent concentration in an object under examination after contrast agent administration is provided, said concentration being determined on the basis of acquired MR images that were taken of the object under examination before and after contrast agent administration. According to the automated method, a model equation is determined which describes the contrast agent concentrations as a function of a plurality of model parameters, wherein at least one model parameter is determined from the acquired MR images. In addition, values are determined for the plurality of model parameters taking the acquired MR images into account. The contrast agent concentration as also determined on the basis of the model equation and the determined values and it is checked whether the contrast agent concentration determined is within an expected value range. If this is not the case, a corrected contrast agent concentration is determined on the basis of the model equation and on the basis of a corrected value for at least one of the model parameters. The corrected value for this at least one model parameter is determined such that the contrast agent concentration having the corrected value is within the expected value range.

The corrected value for the at least one model parameter is determined so that physiologically meaningless contrast agent concentrations are eliminated from the outset and, if these concentrations occur, a re-calculation takes place. Instead of setting the concentration to zero, corrected values are calculated for one or more model parameters so that altogether it can still be ascertained that an increased uptake of contrast agent has taken place in a particular area.

For example, the corrected value of the model parameter is determined such that the corrected contrast agent concentration calculated on the basis of the model equation and the corrected value does not become negative.

In addition, the model equation can have a quotient. In this case the corrected value is determined such that the denominator of the quotient is non-negative and non-zero, i.e. is greater than zero.

One of the model parameters is the intensity change in the MR images from a first MR image before contrast agent administration to a second MR image after contrast agent administration. Determining the corrected value for this model parameter can now mean that a maximum intensity change that is expected to occur is determined. The corrected value for the intensity change is now determined such that it is below the maximum intensity change if the intensity change value determined is above the maximum intensity change.

For determining the maximum intensity change, the denominator of the quotient can have first model parameters which include the intensity change. If the remaining values of the first model parameters are known, the maximum intensity change can be determined by determining the value signal change for which the quotient is greater than or equal to zero.

The maximum intensity change can be determined as follows:

$$Δ S = \frac{1 - cos ( θ )}{R 1 cos θ \cdot TR} \qquad ( 3 )$$

where ΔS is the maximum relative intensity change, R1 the T1 relaxivity of the object under examination before contrast agent administration, TR the repetition times of the imaging sequence, and θ the angle that was used for the imaging sequence.

It is also possible for the model equation to include the model parameters of the maximum contrast agent concentration which indicates the concentration level of the contrast agent in a blood vessel into which the contrast agent has been injected. The corrected value of the model parameter can thus be determined taking into account that the contrast agent concentration in the region being examined is less than or equal to the maximum contrast agent concentration as it occurs in the blood vessel into which the contrast agent is injected. It is likewise possible for the T1 relaxivity to be one of the model parameters, wherein the corrected value for the T1 relaxivity is determined. Here a maximum relaxivity is determined and the corrected value for the T1 relaxivity is determined such that it is below the maximum T1 relaxivity if the actually determined value of the T1 relaxivity is above the maximum T1 relaxivity.

For the determinations of the maximum T1 relaxivity, the denominator of the quotient can again have the first model parameters which include the T1 relativity. If the remaining values of the first model parameters are known, the maximum T1 relativity can be determined by determining the values of the T1 relaxivity for which the quotient is greater than or equal to zero.

The maximum relaxivity can be determined using the following equation:

$$R 1 \leq \frac{1 - cos ( θ )}{Δ S ( t ) cos ( θ ) TR} \qquad ( 4 )$$

The model parameters from the model equation for the contrast agent constellation can generally include the following model parameters:

the relative signal change ΔS between the acquired MR images before/after contrast agent administration, the T1 relaxivity R1 of the object under examination before contrast agent administration, the repetition time TR of the imaging sequence, the flip angle θ, and the specific relaxivity of the contrast agent.

Also provided is a method for determining at least one pharmacokinetic parameter of a pharmacokinetic model, wherein the model describes a contrast agent concentration in the object under examination after contrast agent administration as a function of time. MR images are provided that were taken of the object under examination before and after contrast agent administration. The pharmacokinetic model is also determined. For this determination, this model is fitted to signal waveforms produced on the basis of the MR images provided. The pharmacokinetic model has for this purpose at least one model parameter from which at least one other derived parameter is determined. Here fitting can mean solving an optimization problem using a cost function, wherein the cost function has a regularization term which ensures that the parameter derived from the at least one model parameter has a value that is within the expected range.

In this embodiment, instead of the contrast agent concentration, the pharmacokinetic model is first determined directly. For the fitting, a regularization can be incorporated here which ensures that the derived parameter is within an expected range and cannot assume values that are physiologically meaningless.

The derived parameter can be the contrast agent concentration, for example. Here the regularization term can ensure that the contrast agent concentration is lower than or equal to a maximum contrast agent concentration. The regularization term can ensure, for example, that the contrast agent concentration can only assume positive values. Here the model parameter of the pharmacokinetic model can be, for example, a T1 relaxation time in the region of interest in the object under examination, a magnetization or contrast agent related parameter such as K_{Trans}, kₑₚ or the extracellular volume.

In addition, a device is provided which comprises a processor and a memory with control commands, wherein a method as described above is carried out when the control commands are executed in the tester. The features presented above and the features described in the following can not only be used in the corresponding explicitly presented combinations, but also in other combinations, unless explicitly stated otherwise.

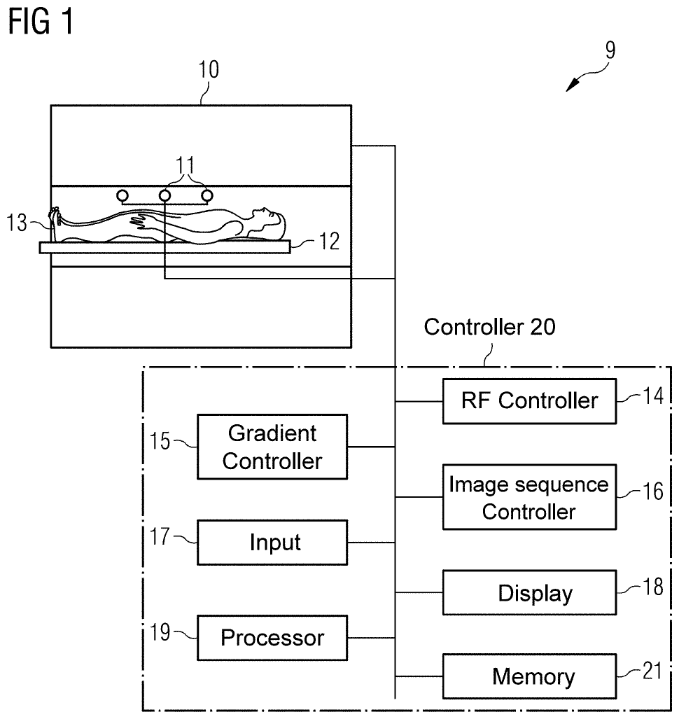

Referring to FIG. 1, an MR system is specifically shown with which it is not only possible to acquire MR images, but it is also possible to use these to determine improved contrast agent concentrations or pharmacokinetic parameters. The MR system 9 comprises a magnet 10 for producing a polarization field BO, wherein a person under examination 13 disposed on a couch 12 is moved into the magnet where position-encoded magnetic resonance signals from the person under examination 13 are received. The coils 11 used for signal reception can be body coils or local coils. Irradiation with RF pulses and switching of magnetic field gradients enables the magnetization produced by the polarization field B0 to be displaced from the equilibrium state and position-encoded, wherein the resulting magnetization can be detected by the receive coils. How MR images can be generated by irradiation with RF pulses and switching of magnetic field gradients in different combinations and sequences is well known to persons skilled in the art and will not be explained in more detail here. In addition, the contrast agent can be injected (not shown) into the person under examination 13, wherein MR images of particular regions of the person under examination before, during and after contrast agent administration can be acquired.

The MR system also has a controller 20 which can be used to control the MR system 9. The controller 20 can have an RF controller 14 for controlling and generating the RF pulses for displacing the magnetization, a gradient controller 15 for controlling and producing the necessary magnetic field gradients. An image sequence controller 16 can control the sequence of magnetic field gradients, signal detection and RF pulses and therefore indirectly the gradient controller 15, the receive coils and the RF controller 14. An operator can control the MR system via an input 17, and MR images or other information necessary for control can be shown on a display 18. A processor 19 comprising at least one processor unit is provided for controlling the different units in the controller 20. Additionally, provided is a memory 21 in which, for example, program modules or programs can be stored which can control the MR system process when they are executed by the processor 19 or more specifically by its processor unit. The processor 19 or rather the entire controller 20 can be designed such that contrast agent concentrations can be calculated in such a way that, among other things, regions in the person under examination 13 having high contrast agent uptake can be reliably detected. In an exemplary embodiment, the controller 20 and/or one or more components therein includes processor circuitry that is configured to perform one or more respective functions and/or operations.

One possibility for preventing physiologically meaningless parameters is to limit the intensity change that is produced in the MR signal before and after contrast agent administration. For example, if the repetition time TR and flip angle are known and only an estimate of the T1 time is available which may be inaccurate, it is possible to limit the intensity change to values that are lower than the following values:

$$Δ S = \frac{1 - cos ( θ )}{R 1 cos θ \cdot TR} \qquad ( 3 )$$

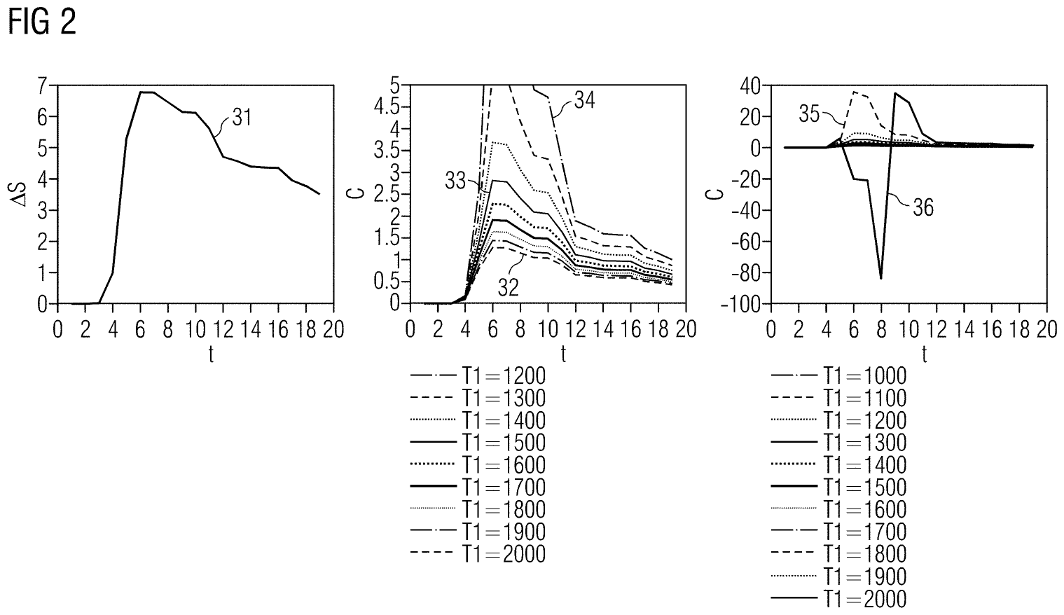

FIG. 2 (left) shows an example of a relative signal change 31 over time after contrast agent administration. If, using this value of the relative signal intensity change, different T1 times are now assumed, it is possible to calculate the contrast agent concentration over time. The dashed line 32 here relates to a T1 time of 2000 ms, the line 33 to a T1 time of 1500 ms, and the dash-dotted line to a T1 time of 1200 ms (line 34). The right-hand diagram shows when lower T1 times are used. The curve 35 relates to a T1 time of 1100 ms and the curve 36 to a T1 time of 1000 ms. Here it can be seen that, for the lower T1 time, the calculated concentration suddenly becomes negative, which is physiologically meaningless. All in all, FIG. 2 indicates that, for T1 times greater than 1200 ms, the results appear meaningful, whereas for lower T1 times, meaningless results are obtained.

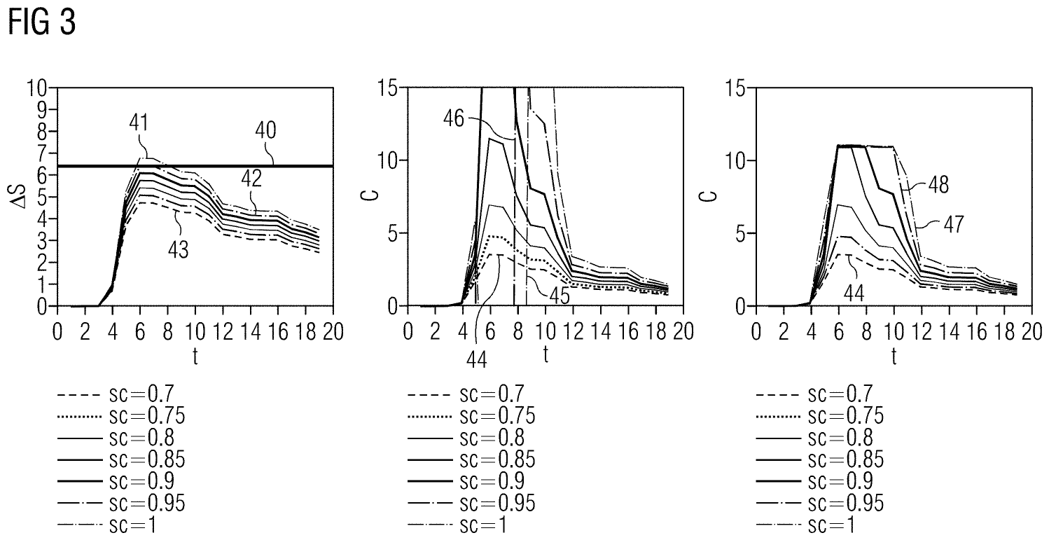

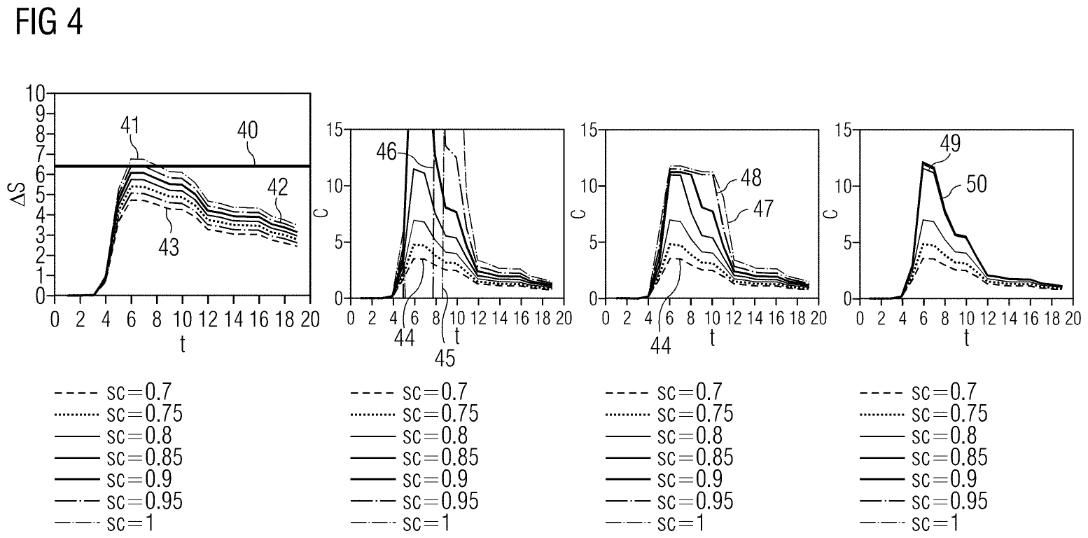

FIG. 3 now shows how this problem can be eliminated by limiting the relative signal change to a maximum value as per the above equation. FIG. 3 (left) shows by way of example the limit 40 as it can be calculated for the maximum intensity change using the above equation (3). For pixels whose signal response is such that the curve is above the limit, e.g. the response curve 41, this would result (center) in a negative concentration, as indicated by the response curve 45 which is shown as a dashed line and, in the central region, clearly drifts into the negative concentration range (not shown). This also applies to the intensity response curve 42 which results in a concentration response curve 46 which likewise has negative regions (shown dash-dotted). FIG. 3 (right) shows the concentration values that result from the signal increase being limited to the maximum values as indicated in FIG. 3 (left). Limiting of the curves 41 and 42 to the limit value 40 produces, instead of the curve 45, the curve 47 and, instead of the curve 46, the curve 48. For an assumed intensity response as shown by the curve 43, a concentration development over time is produced which is identical both in the central and right-hand diagram, as shown by the curve 44. To summarize, this means that the contrast agent constellation therefore remains positive. FIGS. 3 and 4 show different curves for different scaling values sc.

It is likewise possible for the T1 relaxation time or alternatively a scaling factor linked to the B1 inhomogeneity to be adjusted e.g. by solving the above equation (3) for the relaxivity R1. This yields the following equation (4):

$$R 1 \leq \frac{1 - cos ( θ )}{Δ S ( t ) cos ( θ ) TR} \qquad ( 4 )$$

The relaxation curves here run positively or below a given value.

As can be seen in FIG. 3, the maximum value in the intensity change results in a capping in the concentration change as can be seen in FIG. 3 for the curves 47 and 48. However, these curves also show a significant change in the concentration of the contrast agent, which indicates increased perfusion in the region of interest. This means that, as can be seen in FIG. 3 (right), the thus corrected values still reliably indicate that malignant tissue may be present here, as tumor tissue usually has an increased contrast agent uptake.

A similar effect occurs if, instead of a maximum intensity change, the T1 value, i.e. the relaxivity, is considered. As can be seen from equation (4), the concentration curve remains positive if the relaxivity is set lower than the value given in equation (4). This means that the T1 time should be greater than the reciprocal in order to prevent negative concentrations. This is illustrated in FIG. 4. The three graphs on the left were explained above in connection with FIG. 3. On the right-hand side, the contrast agent concentration response curve is shown, taking into account that the T1 time is within a predefined value range, as described above by equation (4). It can again be seen, particularly in curves 49 and 50, that the concentrations do not run into the negative region, but remain in a higher range, as shown by the curves 49 and 50. As can be seen by comparing the graphs 49 and 50 with the graphs 47 and 48, by adjusting or rather limiting the T1 time, i.e. the relaxivity, the shape of the curve is better retained. In the above scenario, conventional processing was used where a T1 card first was created or a global T1 value was used. The concentration was then calculated and the concentration was subsequently fitted to a pharmacokinetic model. In most applications the T1 is also calculated using a gradient echo sequence, often with identical parameters to those in the subsequent contrast agent enhanced measurement wherein, however, different parameters are varied to determine the T1 time, such as e.g. the flip angle.

It is possible to combine the T1 acquisitions and the dynamic contrast agent measurements and/or to combine the fitting of T1 and the pharmacokinetic parameters. Also in this case it is possible to limit the permitted model space to physically plausible scenarios in which the positivity of the relaxation rates are implemented. In a technical implementation this can take place, for example, using so-called dictionary matching in which only physically plausible entries are used. In this case MR signals are synthesized based on the assumed signal modeling for a discrete set of model parameters. Signal waveforms that are deemed to be implausible are discarded. From the discrete set, the signal waveform most closely matching the measured waveform is determined for each voxel. This can take place in the form of a norm or a correlation. Another possibility is not to first calculate the contrast agent concentration as above in order to then calculate the parameters such as perfusion into the tissue, but to calculate the parameters K_{Trans} or kₑₚ or the extracellular volume which are parameters of a pharmacokinetic model and provide information about the tissue.

It is also possible to determine the pharmacokinetic model directly via a fit, wherein for this purpose this model is fitted to signal waveforms produced on the basis of the MR images, e.g. the intensity response over time in particular regions of the person under examination. Initially, for given T1 values a contrast agent concentration C can be calculated which is based on a known mapping or function which exists between T1 and C. For example, it is possible to use the following linearized form:

$$\frac{1}{T 1} = \frac{1}{T 10} + R 1 {C}_{t} \qquad ( 5 )$$

where T1 is the particular T1 at a time t with contrast agent concentration, T10 the T1 time before or without contrast agent, and R1 a contrast agent specific parameter which is known. Cₜ is the contrast agent concentration determined from measurement data at a time t. The measured concentration response as described above can then be fitted by setting up a cost function as follows:

$${L}_{PkM} = {\sum }_{t} {( {C}_{t} - {\tilde{S}}_{t} ( x ) )}^{2} \qquad ( 6 )$$

where L_{PkM} is the cost function of the pharmacokinetic model, Cₜ is the contrast agent concentration determined from the measurements, and \tilde{S}ₜ describes the pharmacokinetic model with the parameters x, wherein the parameters can be multidimensional and stand for the different parameters such as K_{Trans} or V_{E}. t is the index for the measured times.

If flip angle measurements are available, the signal waveforms together with the flip angle measurements can be fitted to a modeling without the concentration being explicitly determined in an intermediate step. A cost function can be set up which simultaneously enforces coincidence with the flip angle measurements and with the contrast agent enhanced measurements.

In the simplest case, for this purpose the T1 change in equation (5) is inserted in the signal equation of the sequence,

thus yielding the following equation:

$${{S}^{\approx }}_{( α , TR )} ( T 10 , M , C ) = M sin α \frac{1 - {e}^{- \frac{TR}{T 10} - \frac{R 1 {C}_{t}}{T 10}}}{1 - cos α {e}^{- \frac{TR}{T 10} - \frac{R 1 {C}_{t}}{T 10}}} \qquad ( 7 )$$

The data can then be simultaneously fitted as follows:

$$L = {\sum }_{n} {( {{S}^{\approx }}_{( n )} ( T 10 , M , C ) - {D}_{n} )}^{2} \qquad ( 8 )$$

wherein L is again the cost function and the pharmacokinetic model \tilde{S}ₜ is used for C. The above summation covers both the flip angle measurements and the contrast agent dynamics, wherein weighting factors can be used. n is the multi-index for the flip angle α, the repetition time TR and the measured times. A regularization term can now be introduced which ensures that the contrast agent concentration remains positive:

\tilde{L}=L+λR (9)

where λ denotes the regularization strength. The regionalization term can be, for example, an entropy such as, for example, a Shannon entropy as shown by the following equation:

$$R ( T 10 , M , \vec{x )} = {\sum }_{t} ( {\tilde{S}}_{t} ( \vec{x )} - 1 - {\tilde{S}}_{t} ( \vec{x )} ln {\tilde{S}}_{t} ( x \vec{)} ) \qquad ( 10 )$$

This formulation enforces in particular the positivity of the concentration.

FIG. 5 recapitulates the first possibility explained. In a step S61 a model equation is set up which describes the contrast agent concentration as a function of the plurality of model parameters, as can be seen in the above equation (1). This model equation has, for example, the model parameters ΔS, R, the flip angle and the repetition time. The values for the model parameters are then determined taking into account the acquired MR images in step S62 and, on the basis of the values obtained, a contrast agent concentration is calculated according to equation (1) in step S63. Here it is checked whether the contrast agent concentration is within a physiologically meaningful range in step S64. If this is not the case, in step S65 at least one corrected model parameter can be determined, i.e. a corrected value for a model parameter is determined. In the example given above, this was either a corrected value for the intensity change or a corrected value for the T1 time. The corrected values are now determined, e.g. using the above equations (2) to (4), so as to ensure that the re-determined concentration having the corrected values is within an expected range.

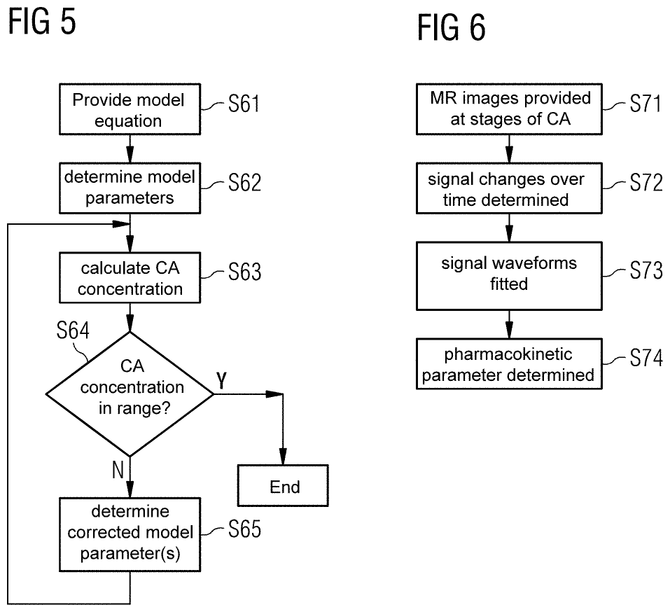

In FIG. 6 another method is summarized in which the pharmacokinetic parameters are determined directly from the pharmacokinetic model, wherein a regularization term is used for the fit so that parameters derived from the model, such as the concentration, are within an expected range. For this purpose, in a step S71, the MR images are provided which were produced before, during and after contrast agent administration. The resulting signal changes over time can now be determined by a pharmacokinetic model which is determined in step S72. In step S73, a fit then takes place to the signal waveforms using the regularization term as described above. The pharmacokinetic model can have the model parameters such as the T1 relaxation or the magnetization or the parameters relevant for dynamic change such as K_{Trans} or kₑₚ. The result of the fit then yields in step S74 the desired pharmacokinetic parameter.

Using the examples described above, it is possible to generate parameter cards of a pharmacokinetic parameter such as K_{Trans} of a model, e.g. a Tofts model, which prevents inhomogeneities that would result from the malfunctioning calculation of the contrast agent concentration which would arise without using the disclosure.

Some pharmacokinetic models such as the Tofts model are based on vascular input functions. Physiologically, it is to be expected that the contrast agent concentration in the tissue will be lower than the contrast agent concentration in the vessel into which the contrast agent was injected. The maximum contrast agent concentration in the vessel can therefore be used as an upper limit when the contrast agent concentration in the tissue is estimated. Here, for example, an upper limit for the corresponding relative signal change can be determined in the above equation (1) when it is solved for AS.

Any connection or coupling between functional blocks, devices, components of physical or functional units shown in the drawings and described hereinafter may be implemented by an indirect connection or coupling. A coupling between components may be established over a wired or wireless connection. Functional blocks may be implemented in hardware, software, firmware, or a combination thereof.

References in the specification to “one embodiment,” “an embodiment,” “an exemplary embodiment,” etc., indicate that the embodiment described may include a particular feature, structure, or characteristic, but every embodiment may not necessarily include the particular feature, structure, or characteristic. Moreover, such phrases are not necessarily referring to the same embodiment. Further, when a particular feature, structure, or characteristic is described in connection with an embodiment, it is submitted that it is within the knowledge of one skilled in the art to affect such feature, structure, or characteristic in connection with other embodiments whether or not explicitly described.

The exemplary embodiments described herein are provided for illustrative purposes, and are not limiting. Other exemplary embodiments are possible, and modifications may be made to the exemplary embodiments. Therefore, the specification is not meant to limit the disclosure. Rather, the scope of the disclosure is defined only in accordance with the following claims and their equivalents.

Embodiments may be implemented in hardware (e.g., circuits), firmware, software, or any combination thereof. Embodiments may also be implemented as instructions stored on a machine-readable medium, which may be read and executed by one or more processors. A machine-readable medium may include any mechanism for storing or transmitting information in a form readable by a machine (e.g., a computer). For example, a machine-readable medium may include read only memory (ROM); random access memory (RAM); magnetic disk storage media; optical storage media; flash memory devices; electrical, optical, acoustical or other forms of propagated signals (e.g., carrier waves, infrared signals, digital signals, etc.), and others. Further, firmware, software, routines, instructions may be described herein as performing certain actions. However, it should be appreciated that such descriptions are merely for convenience and that such actions in fact results from computing devices, processors, controllers, or other devices executing the firmware, software, routines, instructions, etc. Further, any of the implementation variations may be carried out by a general-purpose computer.

For the purposes of this discussion, the term “processor circuitry” shall be understood to be circuit(s), processor(s), logic, or a combination thereof. A circuit includes an analog circuit, a digital circuit, state machine logic, data processing circuit, other structural electronic hardware, or a combination thereof. A processor includes a microprocessor, a digital signal processor (DSP), central processor (CPU), application-specific instruction set processor (ASIP), graphics and/or image processor, multi-core processor, or other hardware processor. The processor may be “hard-coded” with instructions to perform corresponding function(s) according to aspects described herein. Alternatively, the processor may access an internal and/or external memory to retrieve instructions stored in the memory, which when executed by the processor, perform the corresponding function(s) associated with the processor, and/or one or more functions and/or operations related to the operation of a component having the processor included therein.

In one or more of the exemplary embodiments described herein, the memory is any well-known volatile and/or non-volatile memory, including, for example, read-only memory (ROM), random access memory (RAM), flash memory, a magnetic storage media, an optical disc, erasable programmable read only memory (EPROM), and programmable read only memory (PROM). The memory can be non-removable, removable, or a combination of both.

1. A method for determining a contrast agent concentration in an object under examination after contrast agent administration on the basis of acquired magnetic resonance (MR) images taken of the object under examination before and after contrast agent administration, the method comprising:
determining a model equation describing the contrast agent concentration as a function of a plurality of model parameters, at least one model parameter being determined from the acquired MR images;
determining values for at least one model parameter based on the acquired MR images;
determining the contrast agent concentration based on the model equation and the determined values for the at least one model parameter;
checking whether the determined contrast agent concentration is within an expected value range; and
in response to the determined contrast agent concentration being outside the expected value range, determining a corrected contrast agent concentration based on the model equation and a corrected value for at least one of the plurality of model parameters, wherein the corrected value for the at least one model parameter of the plurality of model parameters is determined such that the contrast agent concentration having the corrected value is within the expected value range.

2. The method as claimed in claim 1, wherein the corrected value of the model parameter is determined such that the corrected contrast agent concentration that is calculated based on the model equation and the corrected value does not become negative.

3. The method as claimed in claim 2, wherein the model equation has a quotient, the corrected value being determined such that a denominator of the quotient is greater than or equal to zero.

4. The method as claimed in claim 1, wherein one of the plurality of model parameters is an intensity change in the MR images, the intensity change being a change in signal intensity from a first MR image acquired before contrast agent administration to a second MR image acquired after contrast agent administration, wherein the determining of the corrected value includes determining a maximum intensity change, a corrected value for the intensity change, which is below the maximum intensity change, is determined in response to the determined intensity change value exceeds the maximum intensity change.

5. The method as claimed in claim 4, wherein, for determining the maximum intensity change, a denominator of a quotient of the model equation has first model parameters which include the intensity change, wherein, in response to remaining values of the first model parameters being known, the maximum intensity change is determined by determining signal change values for which the quotient is greater than or equal to zero.

6. The method as claimed in claim 5, wherein the maximum intensity change is determined according to the following equation:
$$Δ S ( t ) = \frac{1 - cos ( θ )}{R 1 cos θ \cdot TR}$$
where ΔS is a relative intensity change, R1 is the T1 relaxivity of the object under examination before contrast agent administration, TR is the repetition time of an imaging sequence with which the MR images before and after contrast agent administration were created, and θ is the flip angle used for the imaging sequence.

7. The method as claimed in claim 1, wherein one of the plurality of model parameters is a T1 relaxivity of the object under examination before contrast agent administration, determination of the corrected value for the T1 relaxivity is performed and includes determining a maximum relaxivity, wherein the corrected value for the T1 relaxivity, which is below the maximum T1 relaxivity, is determined in response to the determined value of the T1 relaxivity being above the maximum Ti relaxivity.

8. The method as claimed in claim 7, wherein, for the determination of the maximum T1 relaxivity, a denominator of a quotient of the model equation has first model parameters which include the T1 relaxivity, wherein, in response to remaining values of the first model parameters being known, the maximum T1 relaxivity is determined by determining T1 relaxivity values for which the quotient is greater than or equal to zero.

9. The method as claimed in claim 8, wherein the maximum relaxivity is determined using the following equation:
$$R 1 \leq \frac{1 - cos ( θ )}{Δ S ( t ) cos ( θ ) TR}$$
where ΔS is a relative signal change between the acquired MR images produced after and before contrast agent administration, R1 is the T1 relaxivity of the object under examination before contrast agent administration, TR is the repetition time of an imaging sequence with which the MR images were produced before and after contrast agent administration, and θ is the flip angle used for the imaging sequence.

10. The method as claimed in claim 1, wherein the model equation includes model parameter of a maximum contrast agent concentration indicative of how high a concentration of the contrast agent is in a blood vessel into which the contrast agent was injected, the corrected value of the model parameter being determined based on the contrast agent concentration in the object under examination being lower than or equal to the maximum contrast agent concentration.

11. The method as claimed in claim 1, wherein the plurality of model parameters include at least one of the following model parameters:
a relative signal change ΔS between the acquired MR images after and before contrast agent administration, a T1 relaxivity R1 of the object under examination before contrast agent administration, a repetition time TR of an imaging sequence with which the MR images were produced before and after contrast agent administration, a flip angle used for the imaging sequence, and a specific relaxivity of the contrast agent R1.

12. A method for determining at least one pharmacokinetic parameter of a pharmacokinetic model that describes a contrast agent concentration in an object under examination after contrast agent administration as a function of time, the method comprising:
providing magnetic resonance (MR) images that were taken of the object under examination before and after contrast agent administration; and
fitting the pharmacokinetic model to signal waveforms produced based on the provided MR images to determine the pharmacokinetic model, wherein the pharmacokinetic model includes at least one model parameter and, from the at least one model parameter, a derived parameter is determined, and wherein the fitting includes solving an optimization problem using a cost function including a regularization term that ensures that the derived parameter from the at least one model parameter has a value that is within an expected value range.

13. The method as claimed in claim 12, wherein the parameter derived from the at least one model parameter is the contrast agent concentration, the regularization term ensuring that the contrast agent concentration is lower than or equal to a maximum contrast agent concentration.

14. The method as claimed in claim 12, wherein the parameter derived from the at least one model parameter is the contrast agent concentration, the regularization term ensuring that the contrast agent concentration only assumes positive values.

15. The method as claimed in claim 12, wherein the at least one model parameter includes at least one of the following parameters: a T1 relaxation time in a region of interest of the object under examination, a magnetization, and a contrast agent related parameter.

16. The method as claimed in claim 12, wherein the regularization term includes an entropy.

17. A computer program product having a computer program which is directly loadable into a memory of a controller of a magnetic resonance (MR) system, when executed by the controller, causes the controller to perform the method as claimed in claim 1.

18. A non-transitory computer-readable storage medium with an executable program stored thereon, that when executed, instructs a processor to perform the method of claim 1.

19. A controller for determining a contrast agent concentration in an object under examination after contrast agent administration on the basis of acquired magnetic resonance (MR) images that were taken of the object under examination before and after contrast agent administration, the controller comprising:
a memory storing control command; and
a processor configured to execute the control commands stored in the memory to:
determine a model equation describing the contrast agent concentration as a function of a plurality of model parameters, at least one model parameter being determined from the acquired MR images;
determine values for at least one model parameter based on the acquired MR images;
determine the contrast agent concentration based on the model equation and the determined values for the at least one model parameter;
check whether the determined contrast agent concentration is within an expected value range; and
in response to the determined contrast agent concentration being outside the expected value range, determine a corrected contrast agent concentration based on the model equation and a corrected value for at least one of the plurality of model parameters,
wherein the corrected value for the at least one model parameter of the plurality of model parameters is determined such that the contrast agent concentration having the corrected value is within the expected value range.

20. A magnetic resonance (MR) system comprising:
the controller of claim 19; and
a MR scanner configured to acquire the MR images of the object under examination.
